(19) United States
(12) Patent Application Publication
(10) Pub. No.: US 2004/0115164 A1
(43) Pub. Date: Jun. 17, 2004

(54) Soft filament occlusive device delivery system

(76) Inventors: Ryan K. Pierce, Mountain View, CA (US); Ivan Sepetka, Los Altos, CA (US); Maria Aboytes, East Palo Alto, CA (US); Ron French, Santa Clara, CA (US); Kirsten Valley, Mountain View, CA (US); John Miller, Redwood City, CA (US)

Correspondence Address: E. Thomas Wheelock, Morrison & Foerster LLP, 755 Page Mill Road, Palo Alto, CA, 94304-1018, US

(21) Appl. No.: 10/322,279

(22) Filed: Dec. 17, 2002

(51) Int. Cl. A61K 31/785; A61K 31/765

(52) U.S. Cl. 424/78.35; 424/78.37

(57) ABSTRACT

This is a surgical device. Specifically disclosed is a delivery system for placement of implantable occlusive devices. In particular, the occlusive devices may be placed at selected treatment sites in the vascular system. The major variation described here is a combination of occlusive filaments produced from gel polymers and various grippers, engagers, and couplers that are capable of holding onto the often slippery occlusive devices and of releasing them at desired treatment sites within the human body. Processes for use of such devices are also included.

### FIELD OF THE INVENTION

This is a surgical device. Described is a delivery system for implantable occlusive devices. In particular, the occlusive devices may be placed at selected treatment sites in the vascular system. The major variation described here is a system made up of a combination of occlusive filaments produced from gel polymers and various grippers, engagers, and couplers that are capable of holding onto the often slippery occlusive devices and of releasing them at desired treatment sites within the human body. Processes for use of such devices are also included.

### BACKGROUND OF THE INVENTION

Vaso-occlusive devices are surgical implements or implants that are placed within the vasculature of the human body, typically via a catheter, either to block the flow of blood through a vessel making up that portion of the vasculature by formation of an embolus or to form such an embolus within an aneurysm stemming from the vessel. Other vascular abnormalities treated using such devices include arterio-venous malformations, fistulas, and burst blood vessels. Significantly, abnormal vasculature generated in the process of tumor growth may be treated using these vaso-occlusive devices.

The use of such devices has grown radically outside the use of treatment of the vasculature. Virtually any anatomical fluid vessel or opening has been treated or closed using devices of this type.

There are a variety of materials and vaso-occlusive devices commercially and medically in use. Perhaps the most well known of these devices is the Guglielmi Detachable Coil (GDC) shown in U.S. Pat. Nos. 5,122,136 and 5,354,295, both to Guglielmi et al. These patents and many more that follow it, describe a helically wound coil that is introduced to a treatment site in the body by use of a pusher wire that resembles a standard guide wire. The junction between the pusher wire and the coil is an electrolytically erodible joint that, upon application of a small current, will harmlessly erode in the human body separating the pusher wire from the coil. In overall summary, the procedure utilizing the GDC is this: the coil portion of the device is delivered by a catheter to the treatment site, the electricity is applied, the joint separates, the coil remains in the body forming the desired embolus, and the pusher wire and catheter are retrieved from the body. Many other variations of metallic coils are found in the patent literature and on the commercial marketplace.

Another type of occluding material are the embolic agents that are introduced into the human body in a liquid form where they are transformed either by precipitation from solution (e.g., U.S. Pat. No. 5,925,683 to Park) or by chemical reaction.

Another, more recently developed vaso-occlusive material involves biocompatible polymeric agents that are hydratable or gels. They may be introduced into treatment sites in the body much in the same way that the coils are although they typically must be handled in a somewhat different fashion because of the nature of their makeup. The polymers typically are quite slippery and may be damaged if handled with lack of care and understanding.

### SUMMARY OF THE INVENTION

Described here is system for delivering occlusive components into the body, where the delivery system is made up of 1.) at least one occlusive component made up of at least a polymeric gel, that generally is hydratable and filamentary, and 2.) a delivery component. The delivery component includes an engager that has a couple of functions: first, it is able to maintain the withdrawability of the occlusive component until delivery at a selected treatment site in the body, perhaps by grasping the occlusive component and, secondly, it is able to release and to deliver the occlusive component(s) at that selected treatment site. The structure of the system is such that a variety of treatment sites are accessible, but important sites would certainly be found in the vascular system.

The occlusive components may be made up of polymeric materials such as polyacrylamide, hydrophilic polyacrylonitrile, poly(N-isopropylacrylamine), poly(vinylmethylether), poly(ethylene oxide), poly(vinylalcohol), poly(ethyl (hydroxyethyl) cellulose), poly(2-ethyl oxazoline), polylactide, polyglycolide, poly(lactide-co-glycolide), poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester), polycyanoacrylate, polyphosphazene, polyethyleneoxide, polyethyleneglycol, polyacrylicacid, polyacrylonitrile, polyvinylacrylate, polyvinylpyrrolidone, polyglycolic-lactic acid, their block and random copolymers, and their blends and collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, and their block and random copolymers and their blends.

Additionally, the occlusive components may also contain ancillary materials such as bioactive agents and radio-opacifiers. The bioactive agent acts to provide or to promote a correlating biological activity at the implantation site in the patient. For instance, the bioactive agent may be selected from compositions that occlude blood flow, adhere to the occluder at the implantation site, rebuild damaged vascular walls, inhibit or cause regression of capillary dilation, inhibit or cause regression of arterio-venous malformations, and inhibit or cause regression of tumor growth.

By way of example, the bioactive agent may be selected from the group consisting of protein factors, growth factors, inhibiting factors, endothelization factors, extracellular matrix-forming factors, cell adhesion factors, tissue adhesion factors, immunological factors, healing factors, vascular endothelial growth factors, scarring factors, tumor suppression antigen-binding factors, anti-cancer factors, monoclonal antibodies, monoclonal antibodies against a growth factor, drugs, drug producing cells, cell regeneration factors, progenitor cells of the same type as vascular tissue, and progenitor cells that are histologically different from vascular tissue.

The occlusive components may also comprise a radio-opacifier, e.g., a material that provides visibility of the device under X-ray or other imaging technology such as computer assisted tomography (CT scans), magnetic resonance imaging (MRI's), and fluoroscopy. For instance, a selected radio-opacifier may include a gadolinium-based MRI contrast agent. These agents may include gadopentetate, gadopentetate dimeglumine (Gd-DTPA sold as “Magnevist”), gadoteridol (Gd HP-D03A sold as “ProHance”), gadodiamide (Gd-DTPA-BMA sold as “Omniscan”), gadoversetamide (Gd-DTPA-BMEA sold as “OptiMARK”), Gd-DOTA (sold as “Magnevist” or “lotarem”), Gd-DTPA labeled albumin, and Gd-DTPA labeled dextran. Other suitable fluoroscopic radio-opacifiers include those that are variously soluble in the polymer precursors or the polymer itself, e.g., metrizamide (see, U.S. Pat. No. 3,701,771) or iopromide (see, U.S. Pat. No. 4,364,921—often sold in a dilute form under the tradename “Ultravist”) and solid, powdered materials such as barium sulfate, bismuth trioxide, bismuth carbonate, tungsten metal, and tantalum metal, and the like.

The polymeric material may be selected to cooperate with specific solvents or ionic solutions that, respectively, dissolve or produce a phase change (e.g., gel to sol) in a chosen section of the occlusive component. This selection permits “chemical tailoring” of the occlusive component's length.

The delivery component has an “engager” that may be any of several different variations. For instance, the engager may be 1.) a severing component that physically severs the occlusive filamentary shape, 2.) a squeezing member that severs the occlusive filamentary shape, 3.) a “separation region” that is used to isolate an intermediate portion of the occlusive filamentary shape so that a fluid (a solvent or an ionic fluid that initiates a phase change in the polymer) contacts the intermediate portion of the occlusive filamentary shape and severs it, 4.) an outer tubing member having an interior interference member in the lumen that allows the shank of the occlusive filamentary shape to freely slide through but stops the filament at a cooperating filament interference member until a selected hydraulic pressure is applied, 5.) a proximally located pusher member and a distally located interwoven self-expanding tubular member (a “finger puzzle” type device) that grasps the occlusive filamentary shape when the self-expanding tubular member is within the lumen but self-expands when the grasper is pushed outside of the lumen by the pusher, or 6.) a tubing member having a distally located, duck-bill distendible valve.

Examples of the severing component are devices having a cutting string or loop around the occlusive filamentary shape, perhaps embedded in the wall, perhaps extending distally of the delivery component, that cuts the occlusive filamentary shape when the looped string is pulled proximally. The severing component may also be a cutting wire movable across a lumen containing the occlusive filamentary shape to cut the it perhaps upon inflation of a cooperative, properly positioned balloon.

Examples of a squeezing form of the severing component include inflatable balloons positioned to squeeze and to sever the occlusive filamentary shape within the lumen upon inflation, or cooperating, coaxial wall members forming the wall of the delivery device. The cooperating coaxial wall members may, for instance, have interfitting ridges and recesses, be able to slide axially with respect to each other to cause the interfitting ridges and recesses to interfere with each other and squeeze the occlusive filamentary shape.

Examples of an engager that use a separation region to isolate and to allow dissolution or phase change of an intermediate portion of the occlusive filamentary shape and release a distal portion of that occlusive filamentary shape include coaxially arranged inner and outer tubing members. In this example, the outer tubing member has a distal end that seals against the occlusive filamentary shape allowing the annular space between them to provide the active fluid against the occlusive filamentary shape only in the region distal of the inner tubing. The occlusive filamentary shape is severed in that region. In conjunction with this example, the occlusive filamentary shape may have a region of enhanced susceptibility to the fluid (perhaps bracketed by radio-opaque markers) or the occlusive filamentary shape may have a consistent composition.

Another example of the delivery component includes a lumen for maintaining the occlusive filamentary shape at a position against a wall having a fluid access opening. The fluid access opening further communicates with an independent fluid lumen for the severing fluid.

Finally, of this variation of the system, the delivery component may utilize an inflatable balloon member that is positionable within a tubing member lumen to press the occlusive filamentary shape against the wall of the lumen to isolate the lumen proximal of the balloon and to permit introduction of a solvent or ionic solution to presence the occlusive filamentary shape. The solvent or ionic solution is then positioned to sever the filament.

Another example of the invention having a physical engager involves a cooperation between an interference member or region on the occlusive filamentary shape itself and an interference member or region on the interior of a lumen in the delivery component. Generally, the engager may be made up of outer tubing member having an interior, female, distally located interference member with a passageway. That interference member has a size selected to allow the shank of the occlusive filamentary shape to freely slide through. The filament interference member does not pass through, however, until a selected higher hydraulic pressure is applied.

The interference member on the filament may be of any of a variety of shapes and structures. Examples include a widened region of the occlusive filamentary shape, at least one added band, a knot in the occlusive filamentary shape, a helically wound wire or ribbon (perhaps radio-opaque), a widened region of the occlusive filamentary shape, and a spherical member. In some variations, the filament may be severed by a pull from the proximal end, if the filament has been properly narrowed. Another example of a simple engager is a tubing member having an interior lumen sized to fit over and to grasp the occlusive filamentary shape but to allow passage of the occlusive filamentary shape upon application of a selected hydraulic pressure.

The system engager may include a proximally located pusher member designed to eject occlusive filamentary shape from a cooperative portion of the grasper that is distally located and is an interwoven, self-expanding tubular member adapted to grasp the occlusive filamentary shape when the self-expanding tubular member is situated within the outer tubing member lumen and then to self-expand when pushed outside the outer tubing member lumen by the pusher. The self-expanding tubular member may be made of a metal or alloy. Examples of such materials include alloys such as the stainless steels and superelastic alloys such as nitinol.

Finally, the delivery component may be made of a tubing member with a lumen having a distal end and the occlusive filamentary shape is situated within the tubing member lumen and is pushed outside of tubing member lumen by application of hydraulic pressure to the proximal end of the tubing member. A useful addition to such a delivery component tubing member is a distally located, duck-bill distendible valve of a size and strength to allow the occlusive filamentary shape to pass through it and out of the tubing member lumen upon appropriate application of hydraulic pressure.

### BRIEF DESCRIPTION OF THE DRAWINGS

FIG. 1 shows, in perspective, a typical catheter assembly having the occlusive component sticking out from one end.

FIG. 2 shows, in partial cutaway, the introduction of an occlusive implant into an aneurysm in the vasculature using a catheter.

FIGS. 3-9 show partial cutaway, side views, of variations of the combination delivery component and occlusive component, each of which may be delivered using hydraulic pressure.

FIGS. 10 and 11 show partial cutaway, side views, of variations of delivery components or couplers that may be used to cut the occlusive component at desired site using a looped string.

FIGS. 12A and 12B show a balloon-actuated delivery component that uses an implanted wire to cut the occlusive device during delivery.

FIGS. 13A, 13B, and 13C show, respectively, a side view of a non-actuated severing delivery device, the actuated device, and an end view of the device. This device severs by squeezing the occlusive component.

FIGS. 14A-14D show a procedure, in partial side view, of a variation of the combination delivery component and occlusive component, that uses a balloon component to isolate a section of occlusive device and dissolve that section for delivery of a distal portion of the original occlusive device.

FIGS. 15 and 16 show, in side view, partial cutaway, delivery components suitable for isolating a section of the occlusive device and severing a section of that occlusive device using a solvent or ionic solution.

FIGS. 17A and 17B show, in two steps, in a procedure for using an occlusive device severing device using a solvent or ionic solution.

FIGS. 18A and 18B show, in partial cross section, a mechanical grasping device for delivering the occlusive component.

FIG. 19A shows a partial side view of a container holding an occlusive device within it. FIG. 19B shows a partial cut away, side view of the distal tip of the container showing the duckbill valve situated there. FIG. 19C shows an end view of the delivery component also showing the duckbill valve.

### DESCRIPTION

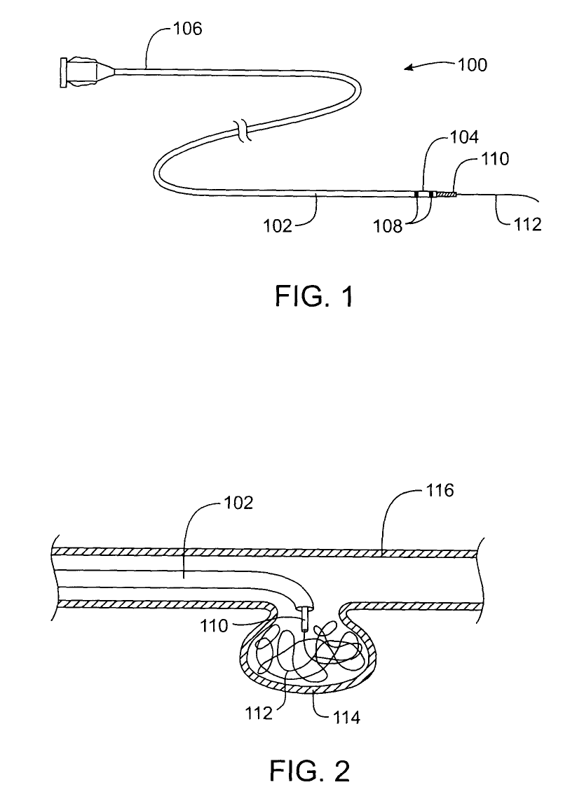

Typically, the occlusive device or component described here will be delivered using a catheter assembly, e.g. (100) as shown in FIG. 1. Catheters are well known devices for delivering occlusive devices into the vasculature. They are thoroughly designed and many variations are available for reaching various regions in the vasculature whether the selected site for treatment be in a large vessel such as the descending aorta or in the fine and narrow vasculature of the brain. Shown in FIG. 1 is a catheter (102) that often is constructed in such a way that the distal end of the catheter (104) is significantly less stiff than the proximal end (106). When the catheter (102) is small, e.g., because it is to be used in the neurovasculature, this is especially true. Also shown in FIG. 1 are radio-opaque markers (108) that allow the end of the catheter to be readily observed using fluoroscopy. The delivery component (110) is also shown as is the filamentary occlusion device (112). The delivery component and the occlusive component will be discussed in more detail below. Of special importance to the description here are the variations in the joint between the two.

FIG. 2 shows the placement of a catheter (102) such as was shown in FIG. 1 as it is used in providing a pathway for the delivery component (110) and the occluding component (112). In FIG. 2, the occlusive component (112) is used to fill an aneurysm (114) that extends from a patent vessel (116).

In general, the occlusive component delivery system described here is made up of a combination of: a.) at least one occlusive component, typically one or more filaments, and typically comprised of a polymeric gel and b.) a delivery component having a grasper, engager, or coupler. The delivery component has the functional task of holding onto the occlusive component until the user, typically a medical doctor, is able to place or situate, the occlusive component at the selected treatment site in the body and then release the occluding component and deliver it to the selected site without a mishap.

By the terms “engager” or “grasper” or “coupler,” we mean a region of a delivery component or a mechanism associated with that delivery component that both a.) maintains the occlusive component under the control of the user to extent that the occlusive component may be removed from or withdrawn from the selected treatment site in the body before that user completes a specific releasing act or acts, e.g., an increase of a hydraulic pressure, a movement of a physical pusher, cutting the occlusive component, etc. and b.) controllably releases that occlusive component and delivers it to the selected treatment site upon completion of that specific releasing act or acts. In addition to the circumstance in which the occlusive component is simply passively held by the “engager” or “coupler,” both the “maintenance under control” and the controllable release of the occlusive component may be the result of the engager's or coupler's relation to a specifically provided cooperative feature of the occlusive component.

This system may deliver one or more occlusive components. Typically, the occlusive components will comprise filamentary shapes. Of particular interest are filaments comprising natural or synthetic polymeric hydratable gel. Synthetic polymers may be, for instance selected from the group consisting of polyacrylamide (PAAM), hydrophilic polyacrylonitrile (HYPAN), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), polylactide (PLA), polyglycolide (PGA), poly(lactide-co-glycolide) PLGA, poly( e-caprolactone), polydiaoxanone, polyanhydride, trimethylene carbonate, poly(β-hydroxybutyrate), poly(g-ethyl glutamate), poly(DTH-iminocarbonate), poly(bisphenol-A iminocarbonate), poly(orthoester) (POE), polycyanoacrylate (PCA), polyphosphazene, polyethylene oxide (PEO), polyethyleneglycol (PEG), polyacrylic acid (PAA), polyacrylonitrile (PAN), polyvinylacrylate (PVA), polyvinylpyrrolidone (PVP), polyglycolic lactic acid (PGLA), their block and random copolymers, and their blends. Natural polymers, for instance, may be materials selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, and their block and random copolymers and their blends. In addition, the occlusive components may contain or be coated with one or more bioactive agents in an amount effective to provide or to promote a selected biological activity and may contain one or more radio-opacifiers.

The bioactive agent typically is selected to provide or to promote a biological activity at the occlusive device's selected implantation site. For instance, the bioactive agent may be selected from the group consisting of compositions that occlude blood flow, adhere to the occlusive device at the site, rebuild damaged vascular wall, regress or inhibit capillary dilation, regress or inhibit venous malformation, and regress or inhibit tumor growth at or near the implantation site.

By way of further example, the bioactive agent may be selected from the group consisting of protein factors, growth factors, inhibiting factors, endothelization factors, extracellular matrix-forming factors, cell adhesion factors, tissue adhesion factors, immunological factors, healing factors, vascular endothelial growth factors, scarring factors, tumor suppression antigen-binding factors, anti-cancer factors, monoclonal antibodies, monoclonal antibodies against a growth factor, drugs, drug producing cells, cell regeneration factors, progenitor cells of the same type as vascular tissue, and progenitor cells that are histologically different from vascular tissue.

The term “an effective amount” a given agent or agents is to be determined on an agent-by-agent basis, taking into account, such standard, known parameters of bioactive agents such as potency, available concentration, and volume of space within the patient to be targeted for the desired effect. Efficacy and proper dosage are determined by routine assays specific for the bioactive agent selected using, for example, standard assays found in well known and frequently used laboratory assay and protocol manuals for identifying activity and quantifying potency of molecules and cells.

The occlusive components may also comprise a radio-opacifier, e.g., a material that provides visibility of the device under X-ray or other imaging technology such as computer assisted tomography (CT scans), magnetic resonance imaging (MRI's), and fluoroscopy. For instance, a selected radio-opacifier may include a gadolinium-based MRI contrast agent. These agents may include gadopentetate, gadopentetate dimeglumine (Gd-DTPA sold as “Magnevist”), gadoteridol (Gd HP-D03A sold as “ProHance”), gadodiamide (Gd-DTPA-BMA sold as “Omniscan”), gadoversetamide (Gd-DTPA-BMEA sold as “OptiMARK”), Gd-DOTA (sold as “Magnevist” or “Iotarem”), Gd-DTPA labeled albumin, and Gd-DTPA labeled dextran. Other iodine-based and powdered metal-based radio-opacifiers are also well-known.

The bioactive agents and radio-opaque materials may be integrated into the typically extruded occlusive components. Integration or inclusion of the bioactive agents and radio-opaque materials into the extruded product may be accomplished during extrusion or after extrusion. Such integration may be accomplished after extrusion such as by the acts consisting of coating, dipping, jacketing, spraying, weaving, braiding, spinning, ion implantation, vapor deposition, and plasma deposition. Integration of the bioactive agents and radio-opaque materials during extrusion may also be accomplished by placing the agent into a solvent used to dissolve the polymeric material making up the occluding filament. The bioactive agents and radio-opaque materials may (depending upon their composition) also be incorporated into the filament during subsequent hydration of the extruded filament.

As will be noted in one or more variations discussed below, the composition of the occlusive component may vary along its length and may well have certain features built into the structure that will cooperate in some fashion to cause or to permit severing the device or releasing it.

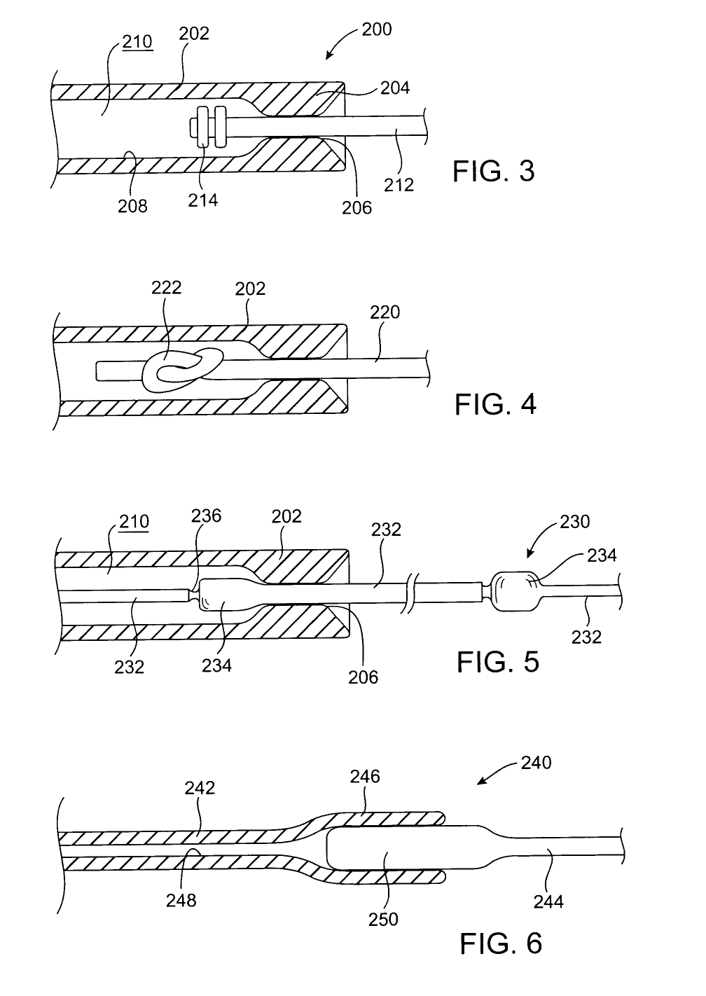

FIG. 3 shows an occlusive component delivery system (200) having a delivery component (202) with an engager section (204). Engager section (204), in this instance, is a region having a smaller inside diameter (206) than its adjacent lumen diameter (208). In this variation (and in many variations discussed elsewhere) the material making up the engager is preferably elastomeric. This allows the diameter (206) to expand when faced with sufficiently increased hydraulic pressure within the chamber or opening (210). The increased pressure (at least when sufficiently increased) causes the interference portions of occlusive device (212) to pass through the female opening or diameter (206) in response to the increased hydraulic pressure in opening (210). In this variation, the filament interference members are a pair of swaged rings (214) mounted on the proximal end of occlusive component (212). It should be pointed out that under modest hydraulic pressure within chamber (210), the shank or shaft of occlusive component (212) will slide easily through diameter (206). The interference rings (214) do not pass through diameter (206) without the presence of a still higher hydraulic pressure in chamber (210).

Similarly, in FIG. 4, delivery component (202) has the same or similar components to that shown in FIG. 3. The difference in this variation is that occlusive component (220) includes a knot (222) found in its proximal end. When the occlusive components found in FIGS. 3 and 4 are delivered, until another is added, there is no more occlusive component to be found within the delivery device (202).

In contrast, the occlusive component (230) found in FIG. 5 has sections of desired length and those sections may be delivered to the selected treatment site, one section at time. Again, delivery component (202) as shown in FIGS. 3 and 4. The occlusive component (230) shown in FIG. 5 has a number of shanks or shafts (232) separated by interference sections (234) that are designed to cooperate with the passageway (206) in the following fashion: at a low hydraulic pressure in chamber (210), the shank of occlusive device (230) is able to slide easily through diameter (206). At a slightly higher pressure, the interference member (234) acts as a “stopper” in diameter (206) but is not ejected. If the user pulls on the proximal end of occlusive component (230), the component (230) should break at the narrowed region (236). Thereafter, at an even higher pressure in chamber (210), the remaining portion of occlusive component (230) should be ejected onto the treatment site.

FIG. 6 shows, in partial cutaway, an occlusive component delivery system (240) having a delivery component (242) and an occlusive component (244). The delivery component (242) has a distal end that is expanded in diameter relative to the diameter of the adjacent lumen (248) in delivery component (242). The expanded diameter (246) fits snugly about the proximal end (250) of occlusive component (244). The proximal end (250) of occlusive component (244) is shown to have a diameter larger than the shank of the occlusive component just adjacent, but need not be so.

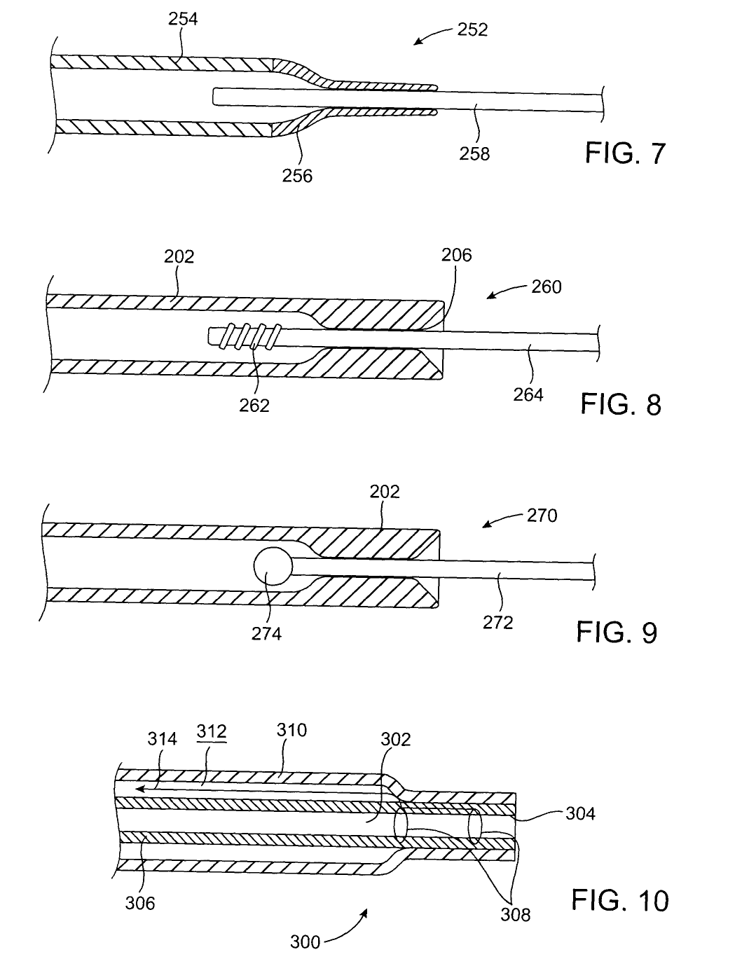

FIG. 7 shows another variation of the occlusive component delivery system (252) having a delivery component (254) with a reduced tip (256) all grasping upon an occlusive implant (258). Desirably, at least the distal tip (256) of delivery component (254) is elastomeric and is easily capable of engaging and holding the occlusive component (258) in the position shown. The distal end region (256) of delivery component (254) may have an adjacent wall (a bit more proximal) made out of the same material or it may be made of another material that is somewhat stiffer. The various walls may also be of a composite nature, e.g., layers of thermoplastic polymers sandwiching a braid or coil, to provide the delivery component with some added measure of stiffness, if so desired.

FIG. 8 shows another variation of an occlusive component delivery system (260) in which the interference member (262) on occlusive component (264) comprises a helical coil, perhaps formed of a radio-opaque wire or ribbon. Although the diameter of the coil (262) shown in FIG. 8 is depicted as being constant, it is within the scope of this description that the diameter of the coil may be wound to vary, preferably in a progressive fashion, to assist in its passage through diameter (206) of delivery component (202).

FIG. 9 shows another variation (270) of the occlusive component delivery system having an occlusive component (272) with a spherical end molded onto or otherwise formed at the proximal end.

As noted above, each of the variants shown in FIGS. 3, 4, 5, 6, 7, 8, and 9 are deliverable by the use of hydraulic pressure. Although many of the variations discussed below can be use in conjunction with a hydraulic stream to displace the occlusive component, the delivery components (at least in FIGS. 10-17B) are also used to sever occlusive components at selected lengths as a part of the delivery.

FIG. 10 shows a delivery component (300) having an internal lumen (302) for passage of a occlusive component therethrough. Embedded in the walls (304) of the interior tubular member (306) are one or more cutting loops (308). An outer tubular member (310) surrounds inner tubular member (306) and forms an annular space (312) through which the string (314) that is attached to cutting loops (308), may pass proximally to the user. As string (314) is pulled, the cutting loop (308) exits the wall (304) and cuts the occlusive component into at least two pieces. The string (314) and its accompanying loop or loops (308) desirably are of a tough and sharp material having a significant amount of flexibility. One such easily obtainable material is expanded polytetrafluoroethylene (ePTFE).

This variation and several of those that follow have the advantage of allowing the placement of a specific length of vaso-occlusive material in or at the desired treatment site. Selection of a proper amount of vaso-occlusive material during a medical procedure is not necessarily a trivial task. Conceptually, it is seen to be easier to adjust the amount of material to be implanted by measurement within the body, than it is to calculate the appropriate length before introducing the material into the selected treatment site.

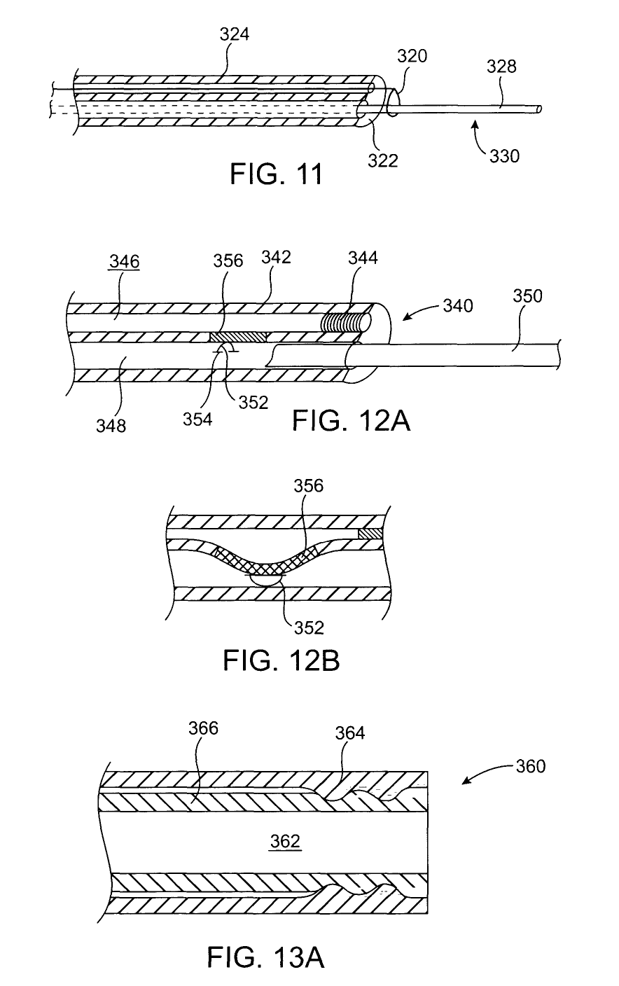

FIG. 11 shows an arrangement similar to that shown in FIG. 10 except that the loop of material or cutting string (320) extends from the distal end (322) of a two lumen catheter shaft (324). Again, a simple tug on the proximal end (326) will cause the cutting loop (320) to sever the more distal section (328) of occlusive component (330).

FIG. 12A shows another variation (340) of the occlusive component delivery system. This variation uses a two lumen catheter shaft (342). One shaft has a distal plug (344) closing lumen (346) and forming, what is essentially, a large, elongate, partially inflatable balloon structure. The delivery component (350) comprises the other lumen (348). Central to this variation is cutting wire (352). Cutting wire (352) is mounted within lumen (348) is a semicircular fashion. The cutting wire (352) may have stabilizer bars (354) at its ends to maintain cutting wire (352) in relatively semi-circumferential position. It is desirable that spring wire (352) be springy and stressed in the position shown. When hydraulic pressure is increased in closed lumen (346), a ballooning wall (356) presses against cutting wire (352) and snaps it “over center” into the position shown in FIG. 12B, thereby cutting any occluding material found in lumen (348).

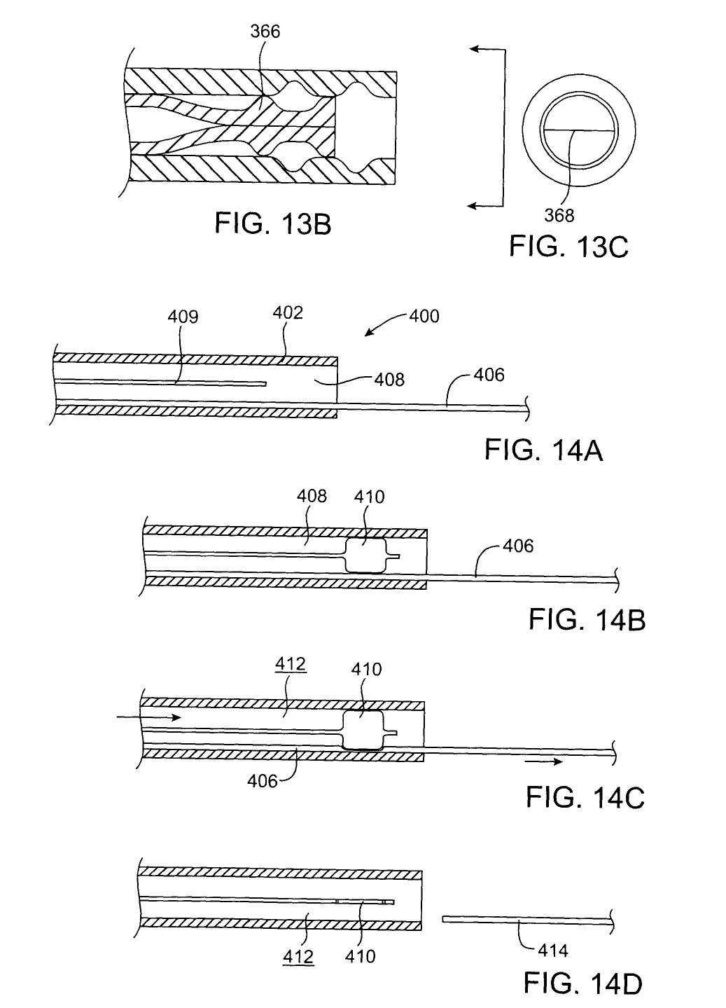

FIGS. 13A, 13B, and 13C show a variation (360) of a delivery component that is adapted to squeeze an occlusive element found within lumen (362). In so squeezing, the soft occlusive component is severed and delivered to the treatment site. The delivery component (360) is made up of an outer tubular member (364) and an inner tubular member (366). The inner member (366) and the outer member (364) are able to slide longitudinally against each other. The combination of “hills” and “bumps” or “ridges” with “valleys,” as they slide against each other, squeezes the inner member (366) as shown in FIG. 13B in such a way that the internal lumen surface of lumen (362) collapses to form a substantially flat or contacting surface (368) as seen in FIG. 13C. The ridges, hills, and valleys shown in partial cross section in 13A and FIG. 13B generally extend only partially around the circumference, but, of course, may extend completely around the circumference.

Many of the polymers listed above, are of the type that are readily dissolved in solvents that are compatible with the human body, for instance, solutions of ethanol or dimethylsulfoxide (DMSO) will dissolve many of the polymers listed above, particularly when care is taken not to select polymers that are cross-linked or have extensive molecular weight. Another methodology for separating continuous fibrillar lengths of polymeric materials into smaller portions for delivery into the selected body site involves the selection of a polymer (for the occlusive component) having the ability to undergo phase transitions from a gel to a sol upon application of a selected ionic solution. One such severable paired or complementary polymer-ionic solution may be a mixture of acrylamide-allyl glucose copolymers and concanavalin A forming the hydrogel polymeric occlusive component and the ionic solution comprising a glucose solution. See, for instance, “Characterization of Glucose Dependent Gel-Sol Phase Transition of the Polymeric Glucose-Concanavalin A Hydrogel System” by Obaidat et al., Pharmaceutical Research, Vol. 13, No. 7. 1996

FIGS. 14A-14D, 15, 16, and 17A and 17B depict delivery components in which solvents or ionic solutions are used to sever the occlusive component at the desired length.

FIG. 14A shows first such variation (400) having an outer polymeric tubing member (402) and a inner balloon member (404). The occlusive component (406) is shown within lumen (408) of outer tubular member (402). When the length for occlusive component (406) has been selected, balloon (410) (as shown in FIG. 14B) is inflated thereby pressing occlusive member (406) against lumen (408) wall and isolating the interior of lumen (408) from the distal end of tubular member (402). A solvent or ionic solution is then passed into lumen (408) to dissolve any occlusive component (406) found in that chamber (412). Balloon (410) is then deflated as shown in FIG. 14D and the distal portion (414) of occlusive component (406) is delivered. It may be desirable to remove the solvent or ionic solution from chamber (412) before deflating balloon (410).

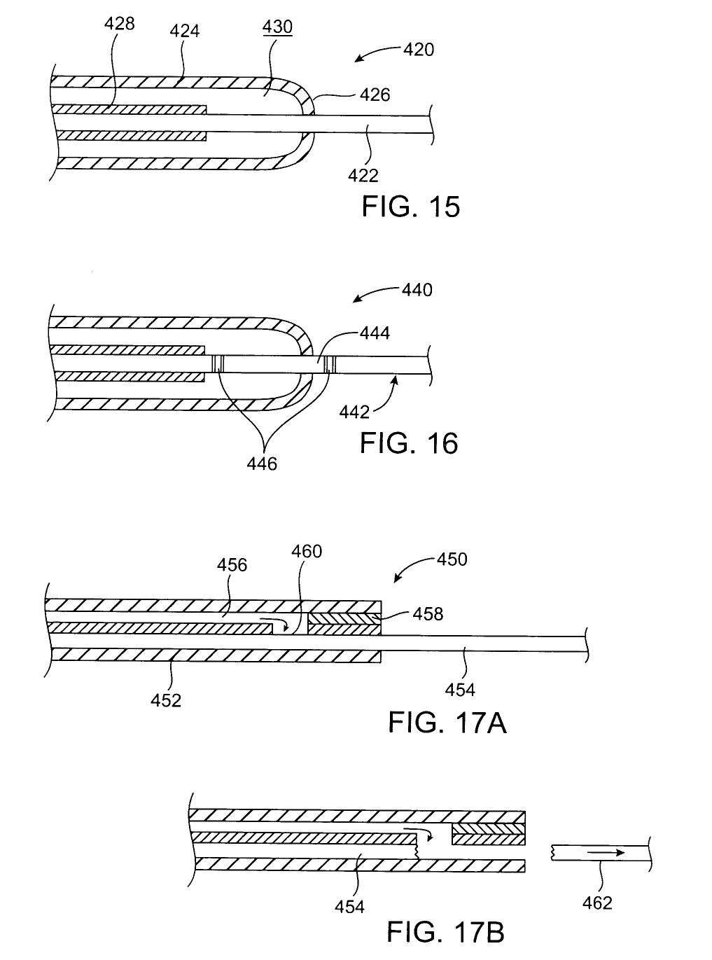

FIG. 15 shows another variation of occlusive component delivery system (420) having a lengthy occlusive component (422). The delivery component has at least two portions: an outer tubular member (424) having distal tip (426) which is nosed down to form a small opening generally matching the size of the occlusive component (422). Within outer tubular member (424) is inner tubular member (428); inner tubular member (428) is provided for the purpose of supporting the occlusive component (422) and shielding a portion of it from a solvent or ionic solution that will be passed through chamber (430) when the size of occlusive component (422) that has passed the distal nose (426) of outer member (424) is appropriate. Solvent or ionic solution is passed through chamber (430). The section of occlusive component (422) that is exposed to the solvent or ionic solution will dissolve thereby releasing the portion of occlusive component (422) that is exterior to nose piece (426).

FIG. 16 shows a variation (440) similar to that found in FIG. 15. However, in this variation, the occlusive component (442) includes a section (or one or more sections) (444) having enhanced solubility characteristics compared to the polymers just adjacent the section (444). The edges of theses regions of superior solubility may be marked by, e.g., radio-opaque marker bands (446) allowing the user having a fluoroscope to determine where to position the regions of enhanced solubility.

FIGS. 17A and 17B show a variation of the delivery system (450) having a dual lumen arrangement. A first lumen (452) designed for carrying the occlusive component (554) may be seen in FIG. 17A. A second lumen, or solvent or ionic solution delivery lumen (456) is distally plugged (458) but retains a window (460) open to the other lumen and to the surface of the occlusive component (554). Placement of solvent or ionic solution in lumen (456) dissolves a small region of the occlusive component allowing a distal portion of occlusive component (462) to leave the delivery component. See FIG. 17B.

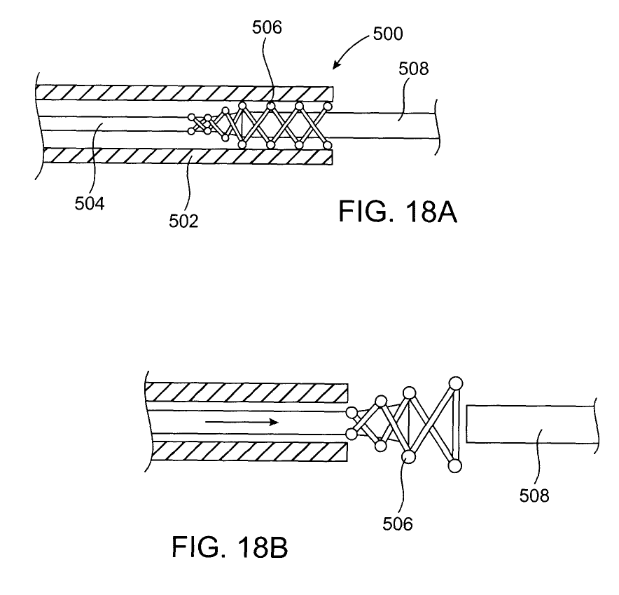

Another variation (500) using a physical grasping device is shown in FIGS. 18A and 18B. Variation (500) includes an outer tubular member (502) and a pusher (504). Mounted distally on pusher (504) is a cage (506) that may be made up of a suitable springy material such as nitinol, stainless steel, or combinations of other superelastic alloys or the like. The cage (506) is preferably of wire or ribbon and, although it may be woven in the same fashion as is a children's finger puzzle, it may also be a pair of counter-wound or co-wound wire or ribbon springs. The cage (506) is formed in such a way that when retracted within tubular member (502), it grasps the occlusive member (508). The cage (506) is self expanding and when it is pushed from the interior lumen of tubular member (502) the diameter expands and the cage releases occlusive member (508) as is shown in FIG. 18B. This is a simple, rugged, and easily understood design for the user.

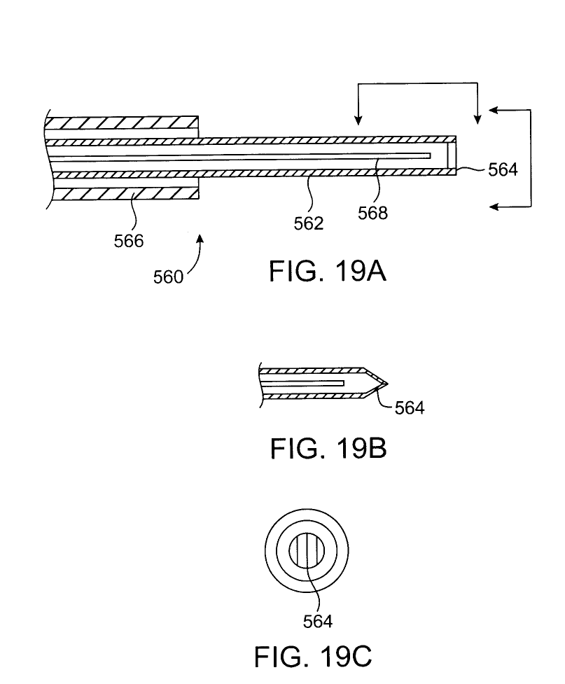

FIG. 19A shows a variation of the occlusive component delivery system (560) and is a simple sheath (562) having, for instance, a duckbill valve (564) shown with more clarity in the sideview, cross section in FIG. 19B and in the end view 19C. Catheter (566) is also shown in FIGS. 19A and 19C.

In this variation, the sheath or sack (562) is simply used as a carrier for the occluding member (568). When the distal end of the carrier (562) is extended to the treatment site, fluid is introduced into the proximal end of carrier (562). With the added flow of fluid, duckbill valve (564) will open and the occlusive component will flow and pass through duckbill valve (564) into or onto the selected treatment site. Duckbill valve (564) may also be used to cut the filamentary occlusive component if so desired.

We claim as our invention:

1. An occlusive component delivery system comprising:
a.) at least one occlusive component comprising at least a polymeric gel, and
b.) a delivery component having an engager that is i.) adapted to maintain the withdrawability of the at least one occlusive component until delivery at a selected treatment site in the body and ii.) adapted to release and to deliver the at least one occlusive component at that selected treatment site in the body.

2. The system of claim 1 wherein the system is adapted to deliver the at least one occlusive components to a vascular site.

3. The system of claim 1 wherein the one or more occlusive components comprise filamentary shapes.

4. The system of claim 3 wherein the one or more occlusive filamentary shapes comprise hydratable gel.

5. The system of claim 1 wherein the one or more occlusive components comprise materials selected from the group consisting of polyacrylamide, hydrophilic polyacrylonitrile, poly(N-isopropylacrylamine), poly(vinylmethylether), poly(ethylene oxide), poly(vinylalcohol), poly(ethyl (hydroxyethyl) cellulose), poly(2-ethyl oxazoline), polylactide, polyglycolide, poly(lactide-co-glycolide), poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester), polycyanoacrylate, polyphosphazene, polyethyleneoxide, polyethyleneglycol, polyacrylicacid, polyacrylonitrile, polyvinylacrylate, polyvinylpyrrolidone, polyglycolic-lactic acid, mixtures of acrylamide-allyl glucose copolymers and concanavalin A, their block and random copolymers, and their blends.

6. The system of claim 1 wherein the one or more occlusive components comprise one or more materials selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, and their block and random copolymers and their blends.

7. The system of claim 5 wherein the one or more occlusive components comprise one or more bioactive agents.

8. The system of claim 6 wherein the one or more occlusive components comprise one or more bioactive agents.

9. The system of claim 5 wherein the one or more occlusive components comprise one or more radio-opacifiers.

10. The system of claim 6 wherein the one or more occlusive components comprise one or more radio-opacifiers.

11. The system of claim 3 wherein the engager comprises a severing component adapted to physically sever the occlusive filamentary shape.

12. The system of claim 11 wherein the severing component comprises a string looped around the occlusive filamentary shape forming a cutting loop, and said string extending from a proximal end of delivery component and adapted so that when the looped string is pulled proximally, the cutting loop diminishes in diameter and cuts the occlusive filamentary shape.

13. The system of claim 11 wherein the at least one cutting loop is embedded in a delivery component wall near a distal end of the delivery component.

14. The system of claim 11 wherein the at least one cutting loop extends distally of the delivery component.

15. The system of claim 11 wherein the severing component comprises a cutting wire movable across a lumen of the delivery component and adapted to cut the occlusive filamentary shape upon said movement, said lumen containing the occlusive filamentary shape.

16. The system of claim 15 wherein the severing component further comprises an inflatable balloon positioned to press the cutting wire across the lumen upon inflation of the balloon.

17. The system of claim 11 wherein the severing component comprises a squeezing member adapted to sever the occlusive filamentary shape upon said squeezing the occlusive filamentary shape in a lumen of the delivery component, said lumen containing the occlusive filamentary shape.

18. The system of claim 17 wherein the squeezing member comprises an inflatable balloon positioned to squeeze and to sever the occlusive filamentary shape within the lumen upon inflation of the balloon.

19. The system of claim 17 wherein the squeezing member comprises cooperating coaxial wall members forming the wall of the delivery device, the cooperating coaxial wall members having at least one set of interfitting ridges and recesses, said coaxial wall members being axially slidable with respect to each other, and, when so axially slid, the interfitting ridges and recesses interfere with each other to squeeze and to sever the occlusive filamentary shape within the lumen.

20. The system of claim 3 wherein the engager comprises a separation region adapted to isolate and to allow dissolution or phase change of an intermediate portion of the occlusive filamentary shape and release a distal portion of that occlusive filamentary shape.

21. The system of claim 20 wherein the delivery component comprises a generally coaxially arranged inner tubing member and an outer tubing member, the inner tubing member for holding the occlusive filamentary shape, the outer tubing member extending distally past the inner tubing member and having a distal end for sealing against the occlusive filamentary shape and forming a space for passing a solvent or ionic solution to the occlusive filamentary shape and severing the occlusive filamentary shape.

22. The system of claim 21 wherein the at least one occlusive filamentary shape includes at least one region of enhanced solubility for a chosen solvent or of enhanced susceptibility to phase change upon application of a selected ionic solution relative to other portions of the at least one occlusive filamentary shape.

23. The system of claim 22 further comprising radio-opaque markers bracketing the at least one region of enhanced solubility of the at least one occlusive filamentary shape.

24. The system of claim 20 wherein the delivery component comprises an occlusive filamentary shape lumen for holding the occlusive filamentary shape, the occlusive filamentary shape lumen having a wall with a fluid access opening, the delivery component further comprising a fluid lumen for passing a solvent or ionic solution to the occlusive filamentary shape at the fluid access opening and severing the occlusive filamentary shape.

25. The system of claim 20 wherein the delivery component comprises an outer tubing member with a lumen having an open distal end, and an inflatable balloon member, the inflatable balloon member being positionable within the outer tubing member lumen to press the occlusive filamentary shape against the wall of the lumen upon inflation of the balloon and to isolate the lumen proximal of the balloon and to permit introduction of a solvent or ionic solution to the occlusive filamentary shape and severing it.

26. The system of claim 3 wherein the occlusive filamentary shape has a shank with a substantially constant diameter and a distally located filament interference member and wherein the engager comprises an outer tubing member having an interior, female, distally located interference member with a passageway, the passageway having a size sufficient to allow the shank of the occlusive filamentary shape to freely slide therethrough but not the filament interference member, but the passageway allowing the passage of the filament interference member under a selected hydraulic pressure.

27. The system of claim 26 wherein the distally located filament interference member comprises a widened region of the occlusive filamentary shape.

28. The system of claim 26 wherein the distally located filament interference member comprises at least one added band.

29. The system of claim 26 wherein the distally located filament interference member comprises a knot in the occlusive filamentary shape.

30. The system of claim 26 wherein the distally located filament interference member comprises a helically wound wire or ribbon.

31. The system of claim 26 wherein the a helically wound wire or ribbon is radio-opaque.

32. The system of claim 26 wherein the distally located filament interference member comprises a spherical member.

33. The system of claim 26 wherein the distally located interference member comprises a widened region of the occlusive filamentary shape having a narrowed diameter just proximal of the widened region, the widened region having a size and shape allowing a pressure to force the widened region against the female interference member and a severing of the occlusive filamentary shape at the narrowed diameter by pulling upon the proximal end of the occlusive filamentary shape.

34. The system of claim 3 wherein the occlusive filamentary shape has a diameter and wherein the engager comprises a tubing member having an interior lumen sized to fit over and to grasp the occlusive filamentary shape but to allow passage of the occlusive filamentary shape upon application of a selected hydraulic pressure.

35. The system of claim 34 wherein the distal end of the engager comprises an interior lumen having an inner diameter larger than an adjacent diameter.

36. The system of claim 34 wherein the distal end of the engager comprises an interior lumen having an inner diameter smaller than an adjacent diameter.

37. The system of claim 3 wherein the delivery component comprises an outer tubing member with a lumen having an open distal end and the engager comprises a proximally located pusher member and a distally located interwoven self-expanding tubular member adapted to grasp the occlusive filamentary shape when the self-expanding tubular member is situated within the outer tubing member lumen and to self-expand when the self-expanding tubular member is pushed outside the outer tubing member lumen by the pusher.

38. The system of claim 37 wherein the interwoven self-expanding tubular member comprises a metal or alloy.

39. The system of claim 37 wherein the interwoven self-expanding tubular member comprises a superelastic alloy.

40. The system of claim 39 wherein the interwoven self-expanding tubular member comprises nitinol.

41. The system of claim 3 wherein the delivery component comprises a tubing member with a lumen having a distal end and the occlusive filamentary shape is situated within the tubing member lumen and is pushed outside of tubing member lumen by application of hydraulic pressure to the proximal end of the tubing member.

42. The system of claim 41 wherein the delivery component tubing member further comprises a distally located, duck-bill distendible valve adapted to allow the occlusive filamentary shape to pass outside of the tubing member lumen by application of hydraulic pressure to the proximal end of the tubing member.
